(19) United States
(12) Patent Application Publication
(10) Pub. No.: US 2021/0383307 A1
(43) Pub. Date: Dec. 9, 2021

(54) INFORMATION PROCESSING APPARATUS AND NON-TRANSITORY COMPUTER READABLE MEDIUM

(71) Applicant: FUJIFILM Business Innovation Corp., Tokyo (JP)

(72) Inventor: Kengo TOKUCHI, Kanagawa (JP)

(73) Assignee: FUJIFILM Business Innovation Corp., Tokyo (JP)

(21) Appl. No.: 16/952,025

(22) Filed: Nov. 18, 2020

(30) Foreign Application Priority Data
Jun. 8, 2020 (JP) 2020-099528

(51) Int. Cl. G06Q 10/06; G06Q 10/10; G16H 50/30; G06N 20/00; G05B 19/4155

(52) U.S. Cl. CPC G06Q 10/063112; G06Q 10/06398; G06Q 10/1095; G16H 20/60; G16H 50/30; G06N 20/00; G05B 19/4155; G06Q 10/06315

(57) ABSTRACT

An information processing apparatus includes a processor configured to change an index of evaluation of a person for a task assigned to the person, depending on whether the task is a physical task or an intellectual task, and perform processing based on a determination of the person when the evaluation is evaluation of a non-physical condition of the person or is other than evaluation indicating that the person is not in poor physical condition and is failing to perform the task.

### CROSS-REFERENCE TO RELATED APPLICATIONS

This application is based on and claims priority under 35 USC 119 from Japanese Patent Application No. 2020-099528 filed Jun. 8, 2020.

### BACKGROUND

### (i) Technical Field

The present disclosure relates to an information processing apparatus and a non-transitory computer readable medium.

### (ii) Related Art

Japanese Unexamined Patent Application Publication No. 2016-197386 describes artificial intelligence technology for various purposes. Artificial intelligence provides dynamic reception of information generated from a living environment and formation of memory in a network from the information, acts on emotions and desires, allows energy circulation, has metacognition, distinguishes itself from others, produces utterances, and performs communication based on the interpreted utterances.

Japanese Unexamined Patent Application Publication No. 2007-172426 describes a collaborative work system in which a person is involved in at least a portion of work performed by each of a plurality of apparatuses that perform their respective work. In the collaborative work system, the timings at which one person intervenes on the plurality of apparatuses are set so as not to overlap each other to allow the plurality of apparatuses and at least one person to work together.

### SUMMARY

Aspects of non-limiting embodiments of the present disclosure relate to accomplishment of a task assigned to a person while taking into account the behavior or state of the person.

Aspects of certain non-limiting embodiments of the present disclosure address the features discussed above and/or other features not described above. However, aspects of the non-limiting embodiments are not required to address the above features, and aspects of the non-limiting embodiments of the present disclosure may not address features described above.

According to an aspect of the present disclosure, there is provided an information processing apparatus including a processor configured to change an index of evaluation of a person for a task assigned to the person, depending on whether the task is a physical task or an intellectual task, and perform processing based on a determination of the person when the evaluation is evaluation of a non-physical condition of the person or is other than evaluation indicating that the person is not in poor physical condition and is failing to perform the task.

### BRIEF DESCRIPTION OF THE DRAWINGS

Exemplary embodiments of the present disclosure will be described in detail based on the following figures, wherein:

FIG. 1 is a block diagram illustrating a configuration of an information processing system according to an exemplary embodiment;

FIG. 2 is a block diagram illustrating a configuration of a control apparatus according to the exemplary embodiment;

FIG. 3 illustrates a task management table; and

FIG. 4 is a flowchart illustrating a process according to the exemplary embodiment.

### DETAILED DESCRIPTION

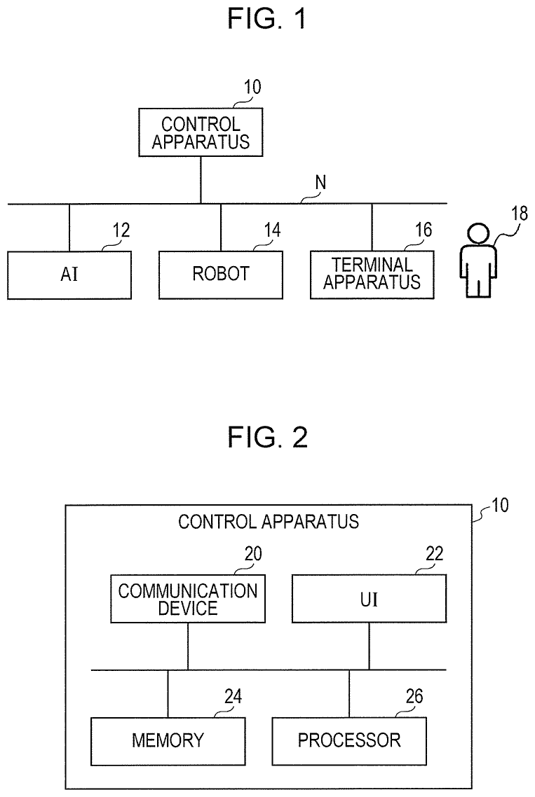

An information processing system according to an exemplary embodiment of the present disclosure will be described with reference to FIG. 1. FIG. 1 illustrates an example configuration of the information processing system according to this exemplary embodiment.

The information processing system according to this exemplary embodiment includes, for example, a control apparatus 10, at least one artificial intelligence (AI) 12, at least one robot 14, and at least one terminal apparatus 16. Any other device (such as a server) may be included in the information processing system.

The terminal apparatus 16 is an apparatus to be used by a person 18. For example, the terminal apparatus 16 may be carried and used by the person 18 or may be placed near the person 18 and used by the person 18.

For example, the AI 12 may be installed in cloud computing or may be installed in a non-cloud computing device such as the robot 14, the terminal apparatus 16, or a server. Specifically, a program implementing the AI 12 may be installed into a device for implementing cloud computing, such as a cloud server, and the AI 12 may be implemented using cloud computing. Alternatively, the program may be installed into a device such as the robot 14, the terminal apparatus 16, or a server, and such a device may implement the AI 12.

At least one of the AI 12 and the robot 14 may be included in the information processing system. That is, both the AI 12 and the robot 14 may be included in the information processing system, or one of the AI 12 and the robot 14 may be included in the information processing system and the other may not be included in the information processing system.

The terminal apparatus 16 may not be included in the information processing system.

The control apparatus 10, the AI 12, the robot 14, and the terminal apparatus 16, which are included in the information processing system, have a function of communicating with any other device. The communication may be wired communication using a cable or wireless communication. That is, the control apparatus 10, the AI 12, the robot 14, and the terminal apparatus 16 may be physically connected to any other device via a cable and transmit or receive information to or from the other device, or may transmit or receive information to or from any other device via wireless communication. Examples of the wireless communication include short-range wireless communication and Wi-Fi (registered trademark). Wireless communication based on any other standard may be used. Examples of the short-range wireless communication include Bluetooth (registered trademark), Radio Frequency Identifier (RFID), and near-field communication (NFC). The control apparatus 10, the AI 12, the robot 14, and the terminal apparatus 16 may communicate with any other device via a communication path such as a local area network (LAN) or the Internet. In the example illustrated in FIG. 1, the control apparatus 10, the AI 12, the robot 14, and the terminal apparatus 16 may communicate with any other device via a communication path N. The communication path N may be the Internet, for example.

In this exemplary embodiment, the person 18 is assigned tasks. The tasks may include either or both of a physical task and an intellectual task.

The category of the concept “physical task” includes, for example, a task involving movement, a task of transporting an object, and a task of processing an object. The physical task may also be a task in which the number of calories consumed by the person 18 per unit time is greater than or equal to a threshold when the person 18 performs the physical task. The calculation of calories and the threshold may be based on the attributes (such as the gender, age, and body type) of the person 18. Instead of or in addition to the number of consumed calories, any other measure, such as the heart rate or the pulse rate, may be used as a reference to define a physical task.

The intellectual task is a non-physical task. The category of the concept “intellectual task” includes operation of a device such as a personal computer (PC) or an electronic device, examples of which include the generation of an electronic mail, posting to a social networking service (SNS), and programming, a conversation with a person or AI, examples of which include a negotiation, a meeting, and a conference, an act of acquiring information, examples of which include reading, viewing information displayed on a display, and listening to audio such as speech or music, and a creative activity. The intellectual task may also be a task in which the number of calories consumed by the person 18 per unit time is less than the threshold when the person 18 performs the intellectual task. Instead of or in addition to the number of consumed calories, any other measure, such as the heart rate or the pulse rate, may be used as a reference to define an intellectual task.

The task assigned to the person 18 is managed by the control apparatus 10 or any other device.

The task may be assigned to at least one of the AI 12 and the robot 14 as a task to be performed in cooperation with the person 18. That is, the person 18 performs the task in cooperation with at least one of the AI 12 and the robot 14. When the task is assigned to at least one of the AI 12 and the robot 14, at least one of the AI 12 and the robot 14 may learn the result of the task if the result is a predetermined good result. For example, at least one of the AI 12 and the robot 14, which has performed the task in cooperation with the person 18, may learn the result of the task, or the AI 12 or the robot 14, which has not performed the task in cooperation with the person 18, may learn the result of the task. The result may also be learned by an external device via the communication path N.

The control apparatus 10 is an apparatus configured to evaluate the behavior of the person 18 for a task assigned to the person 18 and perform processing based on or not based on the determination of the person 18 in accordance with the evaluation. Performing processing not based on the determination of the person 18 refers to, for example, performing processing based on the determination of at least one of the control apparatus 10, the AI 12, and the robot 14. The control apparatus 10 may change the index of evaluation of the person 18 for the task assigned to the person 18, depending on whether the task assigned to the person 18 is a physical task or an intellectual task to evaluate the person 18. The above processes performed by the control apparatus 10 will be described in detail below. The control apparatus 10 corresponds to an example of an information processing apparatus.

The algorithm used for the AI 12 is not limited to any specific one, and any algorithm may be used. Examples of the algorithm include an algorithm for machine learning. The machine learning may be supervised learning, unsupervised learning, or reinforcement learning. Specifically, deep learning (such as multilayer perceptron, convolutional neural network, recurrent neural network, autoencoder, or restricted Boltzmann machine), perceptron, back propagation, Associatron, support vector machine, decision tree, k-nearest neighbor method, linear regression, self-organizing map, Boltzmann machine, principal component analysis, cluster analysis, Q-learning, or the like may be used. An algorithm other than that for machine learning, such as a genetic algorithm or a hill climbing method, may be used. Any other algorithm may be used.

The AI 12 may be installed in the control apparatus 10, the robot 14, the terminal apparatus 16, or the like.

The robot 14 may be, for example, a self-propelled device and is capable of performing a task, a conversation, or the like. The robot 14 may be a humanoid robot, a non-humanoid robot, such as an animal-type robot, or any other robot. The robot 14 includes various sensors such as a camera, a microphone, and an infrared sensor, and is capable of acquiring image data (e.g., still image data or moving image data), audio data, or any other data by using the sensors. Further, the robot 14 is capable of identifying the position of the robot 14, the position of the person 18 around the robot 14, the position of an object around the robot 14 by using Global Positioning System (GPS) or the various sensors. The robot 14 may not be a self-propelled device.

Examples of the terminal apparatus 16 include a PC, a tablet PC, a smartphone, a smart speaker, and a mobile phone. For example, the terminal apparatus 16 may be carried and used by the person 18, or may not be carried by the person 18, but may be placed in position and used by the person 18.

The control apparatus 10 may be incorporated in the robot 14 or the terminal apparatus 16, and the functions of the control apparatus 10 may be implemented by the robot 14 or the terminal apparatus 16. That is, processes performed by the control apparatus 10 may be performed by the robot 14 or the terminal apparatus 16. The functions of the control apparatus 10 may be implemented by the AI 12.

The following describes the hardware configuration of the control apparatus 10 with reference to FIG. 2. FIG. 2 illustrates an example hardware configuration of the control apparatus 10.

The control apparatus 10 includes, for example, a communication device 20, a user interface (UI) 22, a memory 24, and a processor 26.

The communication device 20 is a communication interface having a communication chip, a communication circuit, or the like, and has a function of transmitting information to any other device, and a function of receiving information transmitted from any other device. The communication device 20 may have a wireless communication function or a wired communication function. The communication device 20 may communicate with any other device by using, for example, short-range wireless communication or may communicate with any other device via a communication path such as a LAN or the Internet.

The UI 22 includes a display device and an operation device. Examples of the display device include a liquid crystal display and an electroluminescent (EL) display. Examples of the operation device include a keyboard, an input key, and an operation panel. The UI 22 may be a UI such as a touch panel including both a display device and an operation device. A microphone may be included in the UI 22, or a speaker that emits sound may be included in the UI 22. The UI 22 may not be disposed in the control apparatus 10.

The memory 24 is a device constituting one or more storage areas that store various kinds of information. The memory 24 is, for example, a hard disk drive, various memories (such as a random access memory (RAM), a dynamic RAM (DRAM), and a read-only memory (ROM)), any other storage device (such as an optical disk), or any combination thereof. One or more memories 24 are included in the control apparatus 10.

The memory 24 stores task management information for managing tasks assigned to the person 18, the AI 12, and the robot 14. For example, task operator identification information for identifying a task operator, task information indicating the contents of a task assigned to the task operator, and other related information (such as task date and time information indicating the date and time when the task is performed, and task location information indicating the location where the task is performed) are associated with each other and registered in the task management information. Task operators include at least the person 18 and also include at least one of the AI 12 and the robot 14 that performs the task in cooperation with the person 18. A task may be assigned to a plurality of persons 18. In this case, the plurality of persons 18 may perform the same task in cooperation with each other or may perform the same task separately. Likewise, the same task may be assigned to a plurality of AIs 12 or a plurality of robots 14.

The memory 24 may further store AI management information for managing the AI 12, and robot management information for managing the robot 14.

The AI management information is, for example, information for managing, for each AI 12, information related to the AI 12, and examples of the AI management information include AI identification information for identifying the AI 12, performance information indicating the performance of the AI 12, information indicating tasks that can be performed by the AI 12, and environment information indicating the environment in which the AI 12 operates.

The robot management information is, for example, information for managing, for each robot 14, information related to the robot 14, and examples of the robot management information include robot identification information for identifying the robot 14, performance information indicating the performance of the robot 14, information indicating tasks that can be performed by the robot 14, and environment information indicating the environment in which the robot 14 operates.

At least one of the task management information, the AI management information, and the robot management information may be stored in a device other than the control apparatus 10, such as the robot 14, the terminal apparatus 16, or the server. In this case, the at least one of the task management information, the AI management information, and the robot management information may not be stored in the control apparatus 10.

The processor 26 is configured to control the operation of the components of the control apparatus 10. For example, the processor 26 may communicate with each device by using the communication device 20, display information on the display device of the UI 22, receive information input via the UI 22, store information in the memory 24, or read information from the memory 24. The processor 26 may include a memory.

The processor 26 is also configured to evaluate the behavior of the person 18 for a task assigned to the person 18 and perform processing based on or not based on the determination of the person 18 in accordance with the evaluation. For example, when performing processing not based on the determination of the person 18, the processor 26 controls at least one of the AI 12 and the robot 14 on the basis of the determination of at least one of the processor 26, the AI 12, and the robot 14, and causes at least one of the AI 12 and the robot 14 to perform processing.

The evaluation of the person 18 is obtained, for example, in response to the person 18 failing to perform a task assigned to the person 18. That is, the processor 26 evaluates the behavior of the person 18 when the person 18 is not performing the task assigned to the person 18, and performs processing based on or not based on the determination of the person 18 in accordance with the evaluation. Performing processing not based on the determination of the person 18 refers to, for example, prompting the person 18 to perform the task, or performing an action for encouraging the person 18 to rest. Performing processing based on the determination of the person 18 refers to, for example, not prompting the person 18 to perform the task, or not performing an action for encouraging the person 18 to rest. For example, processing may be performed based on the determination of the person 18, with consideration or respect given to the intention, behavior, or the like of the person 18.

For example, the processor 26 changes the index of evaluation of the person 18 for the task assigned to the person 18, depending on whether the task assigned to the person 18 is a physical task or an intellectual task to evaluate the person 18. Specifically, when the task assigned to the person 18 is a physical task, the processor 26 evaluates the behavior of the person 18 in accordance with the index for physical tasks. When the task assigned to the person 18 is an intellectual task, the processor 26 evaluates the behavior of the person 18 in accordance with the index for intellectual tasks.

For example, if the evaluation of the person 18 is evaluation of a non-physical condition of the person 18 or is other than evaluation indicating that the person 18 is not in poor physical condition and is failing to perform the task assigned to the person 18, the processor 26 may perform processing based on the determination of the person 18. For example, the processor 26 does not cause the AI 12 and the robot 14 to perform processing such as prompting the person 18 to perform the task or encouraging the person 18 to rest.

If the evaluation of the person 18 indicates that the person 18 is in poor physical condition or indicates that the person 18 is not in poor physical condition and is failing to perform the task assigned to the person 18, the processor 26 may perform processing not based on the determination of the person 18. For example, when the person 18 is in poor physical condition, the processor 26 instructs the AI 12 or the robot 14 to contact a medical institution or instructs the robot 14 to encourage the person 18 to rest. When the person 18 is failing to perform the task for a reason other than poor physical condition, the processor 26 instructs the AI 12 or the robot 14 to advise the person 18 to perform the task or instructs the AI 12 or the robot 14 to perform the task on behalf of the person 18.

The processor 26 may perform processing based on the determination of the person 18 when the task assigned to the person 18 is a physical task and the labor time for the task is equal to or longer than a predetermined time.

The processor 26 may prompt the person 18 to perform the task when the task assigned to the person 18 is a physical task, the labor time for the task is less than the predetermined time, and the evaluation of the person 18 does not indicate poor physical condition. For example, the processor 26 instructs the AI 12 or the robot 14 to advise the person 18 to perform the task.

The processor 26 may perform processing based on the determination of the person 18 when the task assigned to the person 18 is a physical task and is a predetermined dangerous task.

The processor 26 may prompt the person 18 to perform the task when the task assigned to the person 18 is a physical task and is not a predetermined dangerous task and the evaluation of the person 18 does not indicate poor physical condition.

The processor 26 may perform processing based on the determination of the person 18 when the task assigned to the person 18 is an intellectual task and is a task of interacting with another person other than the person 18. Examples of the task include a negotiation, a conference, and a meeting.

The processor 26 may prompt the person 18 to perform the task when the task assigned to the person 18 is an intellectual task and is not a task of interacting with another person other than the person 18 and the evaluation of the person 18 does not indicate poor physical condition.

The above processes performed by the processor 26 will be described in detail below.

The processor 26 may assign a task to the person 18 and manage the task. The processor 26 may also assign a task to be performed in cooperation with a person to the AI 12 or the robot 14 and manage the task. For example, the processor 26 manages the task management information described above to manage a task assigned to the person 18, the AI 12, or the robot 14.

Each of the robot 14 and the terminal apparatus 16 includes a communication device, a UI, a memory, and a processor. A device equipped with the AI 12 may include a communication device, a UI, a memory, and a processor.

The following describes the information processing system according to this exemplary embodiment in detail.

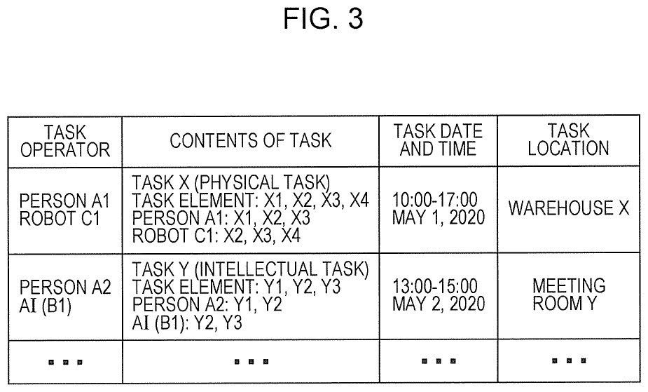

The task management information will be described in detail with reference to FIG. 3. FIG. 3 illustrates an example of a task management table that is an example of the task management information. The data of the task management table is stored in the memory 24 of the control apparatus 10. The device equipped with the AI 12, the robot 14, the terminal apparatus 16, or any other device such as the server may store the data of the task management table.

As illustrated in FIG. 3, in the task management table, for each task operator, a task operator identifier (ID) for identifying the task operator, task information indicating the contents of a task assigned to the task operator, task date and time information indicating the date and time when the task is performed, and task location information indicating the location where the task is performed are associated with each other. The task operator ID is an example of task operator identification information. Examples of the task operator ID include an ID assigned to the person 18, the name of the person 18, an ID assigned to the AI 12, and an ID assigned to the robot 14. The task management table may also be an example of a schedule of tasks.

For example, a task X is assigned to a person A1 and a robot C1. The person A1 is an example of the person 18, and the robot C1 is an example of the robot 14. That is, the person A1 and the robot C1 are assigned the same task X. The task X is assigned to the person A1 and the robot C1 as a task to be performed in cooperation with the person A1 and the robot C1. The task X is a physical task. For example, the task X is a task of transporting objects such as equipment and materials.

The task X includes one or more task elements indicating the respective specific contents of the task X, and the one or more task elements are assigned to the person A1 and the robot C1. The same task element may be assigned to both the person A1 and the robot C1.

For example, the task X includes task elements X1, X2, X3, and X4. The task elements X1, X2, and X3 are assigned to the person A1 as tasks to be performed by a person. The task elements X2, X3, and X4 are assigned to the robot C1 as tasks to be performed by a robot. That is, the task elements X2 and X3 are assigned to both the person A1 and the robot C1. The task elements X2 and X3 are, for example, tasks or the like in which the person A1 and the robot C1 transport an object together. Information indicating the order in which the task elements X1, X2, X3, and X4 are to be performed may be registered in the task management table.

For example, the processor 26 of the control apparatus 10 may assign the task elements X1, X2, and X3 to the person A1 and assign the task elements X2, X3, and X4 to the robot C1. For example, a manager authorized to assign tasks operates the control apparatus 10 or their own terminal apparatus to designate a task operator ID (e.g., the respective task operator IDs of the person A1 and the robot C1), the task elements X1, X2, X3, and X4, which are the specific contents of the task X, the task date and time when the task X is to be performed, such as the start date and time of the task X, the end date and time of the task X, and the duration of the task X, and the task location where the task X is to be performed, and provides an instruction to assign the task X to the person A1 and the robot C1. Upon receipt of the information designated by the manager (e.g., the specific contents of the task X, the task date and time, and the task location) and the instruction, the processor 26 of the control apparatus 10 assigns the task X to the person A1 and the robot C1.

For example, when the manager provides an instruction to assign the task elements X1, X2, and X3 to the person A1 and assign the task elements X2, X3, and X4 to the robot C1, the processor 26 assigns the task elements X1, X2, and X3 to the person A1 and assigns the task elements X2, X3, and X4 to the robot C1 in accordance with the instruction. The processor 26 may refer to the content of the task elements X1, X2, X3, and X4 and assign each task element to the person A1 or the robot C1 in accordance with a predetermined standard (e.g., a standard for assigning a task element to a person, AI, or a robot). For example, tasks suitable for a person to perform, tasks suitable for AI to perform, and tasks suitable for a robot to perform may be defined in advance, and the processor 26 may assign each task element to a person, AI, or a robot in accordance with the definitions.

The processor 26 registers the respective task operator IDs of the person A1 and the robot C1, which are the task operators, task information indicating the task X, the task date and time information, and the task location information in the task management table illustrated in FIG. 3.

Further, the processor 26 notifies the person A1 and the robot C1 of the assignment of the task X to the person A1 and the robot C1. For example, the processor 26 transmits the information on the task X, which is registered in the task management table, to the robot C1 and the terminal apparatus 16 used by the person A1. Specifically, the processor 26 transmits, to the robot C1 and the terminal apparatus 16 used by the person A1, information indicating the assignment of the task X to the person A1 and the robot C1, information indicating the task elements X1, X2, and X3 assigned to the person A1, information indicating the task elements X2, X3, and X4 assigned to the robot C1, task date and time information indicating the task date and time of the task X, and information indicating the task location of the task X. For example, the processor of the terminal apparatus 16 used by the person A1 receives the information described above and causes the display device of the UI of the terminal apparatus 16 to display the information. The robot C1 receives the information described above and recognizes the task X, the task elements assigned to the person A1, the task elements assigned to the robot C1, the task date and time, and the task location.

The processor of the terminal apparatus 16 used by the person A1 may issue an alarm sound, display a message, or turn on a light source at or around the task time.

In another example illustrated in FIG. 3, a task Y is assigned to a person A2 and AI (B1). The person A2 is an example of the person 18, and the AI (B1) is an example of the AI 12. The task Y is assigned to the person A2 and the AI (B1) as a task to be performed in cooperation with the person A2 and the AI (B1). The task Y is an intellectual task. For example, the task Y is a task of negotiating with a person.

Similarly to the task X, the task Y includes one or more task elements indicating the respective specific contents of the task Y.

For example, the task Y includes task elements Y1, Y2, and Y3. The task elements Y1 and Y2 are assigned to the person A2 as tasks to be performed by a person. The task elements Y2 and Y3 are assigned to the AI (B1) as tasks to be performed by AI. That is, the task element Y2 is assigned to both the person A2 and the AI (B1). For example, the task element Y2 is a task or the like in which the person A2 and the AI (B1) manage information.

For example, the processor 26 of the control apparatus 10 may assign the task elements Y1 and Y2 to the person A2 and assign the task elements Y2 and Y3 to the AI (B1). For example, a manager authorized to assign tasks operates the control apparatus 10 or their own terminal apparatus to designate a task operator ID (e.g., the respective task operator IDs of the person A2 and the AI (B1)), the task elements Y1, Y2, and Y3, which are the specific contents of the task Y, the task date and time when the task Y is to be performed, and the task location where the task Y is to be performed, and provides an instruction to assign the task Y to the person A2 and the AI (B1). Upon receipt of the information designated by the manager (e.g., the specific contents of the task Y, the task date and time, and the task location) and the instruction, the processor 26 of the control apparatus 10 assigns the task Y to the person A2 and the AI (B1).

For example, when the manager provides an instruction to assign the task elements Y1 and Y2 to the person A2 and assign the task elements Y2 and Y3 to the AI (B1), the processor 26 assigns the task elements Y1 and Y2 to the person A2 and assigns the task elements Y2 and Y3 to the AI (B1) in accordance with the instruction. The processor 26 may refer to the content of the task elements Y1, Y2, and Y3 and assign each task element to the person A2 or the AI (B1) in accordance with a predetermined standard (e.g., a standard for assigning a task element to a person, AI, or a robot).

The processor 26 registers the respective task operator IDs of the person A2 and the AI (B1), which are the task operators, task information indicating the task Y, the task date and time information, and the task location information in the task management table illustrated in FIG. 3.

Further, the processor 26 notifies the person A2 and the AI (B1) of the assignment of the task Y to the person A2 and the AI (B1). For example, the processor 26 transmits the information on the task Y, which is registered in the task management table, to the AI (B1) and the terminal apparatus 16 used by the person A2. Specifically, the processor 26 transmits, to the AI (B1) and the terminal apparatus 16 used by the person A2, information indicating the assignment of the task Y to the person A2 and the AI (B1), information indicating the task elements Y1 and Y2 assigned to the person A2, information indicating the task elements Y2 and Y3 assigned to the AI (B1), task date and time information indicating the task date and time of the task Y, and information indicating the task location of the task Y. For example, the processor of the terminal apparatus 16 used by the person A2 receives the information described above and causes the display device of the UI of the terminal apparatus 16 to display the information. The AI (B1) receives the information described above and recognizes the task Y, the task elements assigned to the person A2, the task elements assigned to the AI (B1), the task date and time, and the task location.

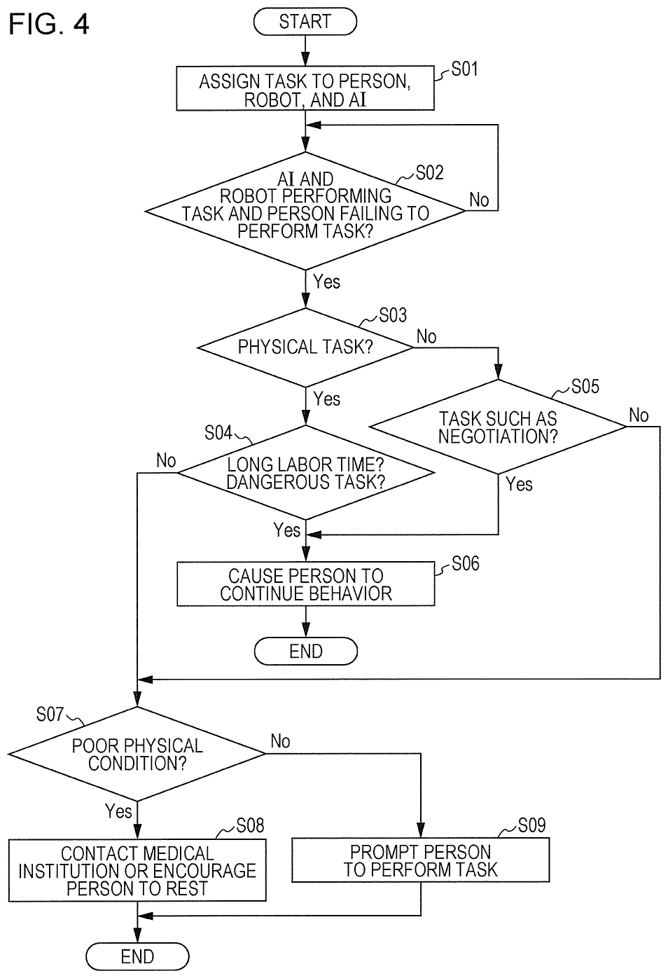

The following describes a process performed by the information processing system according to this exemplary embodiment in detail with reference to FIG. 4. FIG. 4 is a flowchart illustrating the process.

First, the person 18, the AI 12, and the robot 14, which are task operators, are assigned a task (S01). The assigned task and the task operators are registered in, for example, the task management table illustrated in FIG. 3. The assignment of the task may be performed by the control apparatus 10, any other device, the manager, or the like. In one example, a task to be performed in cooperation with a person A3, AI (B3), and a robot C3 is assigned to the person A3, the AI (B3), and the robot C3. The person A3 is an example of the person 18, the AI (B3) is an example of the AI 12, and the robot C3 is an example of the robot 14.

Then, the processor 26 of the control apparatus 10 determines whether the person A3, the AI (B3), and the robot C3, which are assigned the task, are performing the assigned task (S02).

For example, the processor 26 of the control apparatus 10 determines, by using various sensors such as a camera, a microphone, and an infrared sensor, whether the person A3, the AI (B3), and the robot C3 are performing the assigned task. The various sensors may or may not be included in the control apparatus 10. The robot C3 may include the various sensors, and the control apparatus 10 may determine whether the person A3, the AI (B3), and the robot C3 are performing the assigned task, upon receipt of the output of the various sensors of the robot C3.

In a specific example, the person A3, the AI (B3), and the robot C3 are assigned a task Z including task elements Z1, Z2, and Z3, the task element Z1 is assigned to the person A3, the task element Z2 is assigned to the AI (B3), and the task element Z3 is assigned to the robot C3.

For example, the processor 26 of the control apparatus 10 determines, based on information obtained from the various sensors, whether the person A3 is performing the task element Z1. In one example, the task element Z1 may be a physical task of transporting an object in cooperation with the robot C3. In this case, the processor 26 determines, based on image data obtained from the camera or the like, whether the person A3 is transporting the object. For example, if the image data indicates that the person A3 is transporting the object, the processor 26 determines that the person A3 is performing the task element Z1. Otherwise, the processor 26 determines that the person A3 is not performing the task element Z1.

The task element Z1 may be an intellectual task such as a task of operating a PC, a negotiation, a conference, or a meeting. In this case, the processor 26 determines, based on image data obtained from the camera or the like or audio data obtained from the microphone or the like, whether the person A3 is performing the intellectual task. In one example, when the task element Z1 is a task of operating a PC and the image data indicates that the person A3 is operating the PC, the processor 26 determines that the person A3 is performing the task element Z1. Otherwise, the processor 26 determines that the person A3 is not performing the task element Z1. In another example, when the task element Z1 is a negotiation and words related to the negotiation are extracted from audio data indicating the speech of the person A3, the processor 26 determines that the person A3 is performing the task element Z1. Otherwise, the processor 26 determines that the person A3 is not performing the task element Z1.

If the person A3 fails to perform the task element Z1 for a predetermined time or longer, the processor 26 may determine that the person A3 is not performing the task element Z1.

The AI (B3) and the robot C3 may transmit, to the control apparatus 10, information indicating that the AI (B3) and the robot C3 are performing the respective assigned task elements. Upon receipt of the information, the processor 26 of the control apparatus 10 may determine that the AI (B3) and the robot C3 are performing their task elements. The processor 26 may determine, based on the output of the various sensors, whether the AI (B3) and the robot C3 are performing their task elements.

If the person A3 is performing the task element Z1 (No in S02), the processor 26 of the control apparatus 10 is in standby. The processor 26 is also in standby when the person A3 is performing the task element Z1 and the AI (B3) and the robot C3 are not performing the respective assigned task elements.

If the AI (B3) and the robot C3 are performing the respective assigned task elements and the person A3 is not performing the task element Z1 (Yes in S02), the process proceeds to step S03. The process may also proceed to step S03 when the AI (B3) or the robot C3 is not performing the assigned task element and the person A3 is not performing the task element Z1. If the AI (B3) or the robot C3 is not performing the assigned task element, the processor 26 may instruct the AI (B3) or the robot C3 to perform the assigned task element.

In step S03, the processor 26 determines whether the assigned task Z is a physical task.

The processor 26 changes the index of evaluation of the person A3 for the task Z depending on whether the assigned task Z is a physical task or an intellectual task. If the task Z is a physical task, the processor 26 evaluates the behavior of the person A3 in accordance with the index in step S04 or the index in step S07 after step S04. If the task Z is an intellectual task, the processor 26 evaluates the behavior of the person A3 in accordance with the index in step S05 or the index in step S07 after step S05.

If the task Z is a physical task (Yes in S03), the process proceeds to step S04. The following describes the processing of step S04 and the processing after step S04.

For example, if the labor time for the task Z, which is a physical task, is long (Yes in S04), the processor 26 causes the person A3 to continue the current behavior (S06). For example, if the labor time for the task Z is equal to or longer than a predetermined time (Yes in S04), the processor 26 causes the person A3 to continue the current behavior (S06). That is, the processor 26 respects the determination of the person A3 and causes the person A3 to continue the current behavior on the basis of the determination of the person A3. If the labor time for the task element Z1 assigned to the person A3 in the task Z is equal to or longer than the predetermined time, the processor 26 may cause the person A3 to continue the current behavior.

Since the person A3 is not performing the task element Z1 at this stage, the person A3 does not still perform the task element Z1. That is, the processor 26 does not prompt the person A3 to perform the task element Z1. If the task Z is a physical task and if the labor time is equal to or longer than the predetermined time, the person A3 may be resting. Accordingly, the processor 26 does not prompt the person A3 to perform the task element Z1 with consideration of the intention or behavior of the person A3. For example, the processor 26 does not interact with the person A3 to allow the person A3 to continue to rest.

The labor time may be, for example, the length of time elapsed from the start date and time of the task Z or the task element Z1, or may be the length of time during which the person A3 actually performs the task element Z1 (e.g., the total time during which the task element Z1 is performed). In one example, the processor 26 may refer to the task management table illustrated in FIG. 3 or refer to a schedule for managing the task Z to identify the start date and time of the task Z or the task element Z1, and may identify the length of time elapsed from the start date and time. In another example, the processor 26 may cause the camera to capture an image of the person A3 or cause any other sensor to detect the person A3 to actually observe the behavior of the person A3, and may identify the length of time during which the person A3 performs the task element Z1. In still another example, the processor 26 may determine whether the labor time of the person A3 is equal to or longer than the predetermined time, with consideration of the length of time during which the person A3 performs a task other than the task element Z1, the legally prescribed labor time, or the like. For example, if the labor time of the person A3 is equal to or longer than the legally prescribed labor time, the processor 26 does not interact with the person A3.

In another example, if the task Z, which is a physical task, is a predetermined dangerous task (Yes in S04), the processor 26 causes the person A3 to continue the current behavior (S06). If the task element Z1 assigned to the person A3 in the task Z is a predetermined dangerous task, the processor 26 may cause the person A3 to continue the current behavior.

Since the person A3 is not performing the task element Z1 at this stage, the person A3 does not still perform the task element Z1. That is, the processor 26 does not prompt the person A3 to perform the task element Z1. If the task Z is a physical task and is a dangerous task, the person A3 may be careful to take action. Accordingly, the processor 26 does not prompt the person A3 to perform the task element Z1 with consideration of the intention or behavior of the person A3.

In one example, the processor 26 may refer to the task management table illustrated in FIG. 3 or refer to a schedule for managing the task Z to determine whether the task Z or the task element Z1 is a dangerous task. In another example, the processor 26 may observe the behavior of the robot C3 by using the camera or any other sensor to determine whether the task Z is a dangerous task.

If the labor time for the task Z, which is a physical task, is not long (No in S04), the process proceeds to step S07. For example, if the labor time for the task Z is less than the predetermined time (No in S04), the process proceeds to step S07. The process may proceed to step S07 when the labor time for the task element Z1 assigned to the person A3 is less than the predetermined time.

In another example, if the task Z is not a dangerous task (No in S04), the process may proceed to step S07. The process may proceed to step S07 when the task element Z1 is not a dangerous task.

If the labor time for the task Z or the task element Z1 is equal to or longer than the predetermined time or if the task Z or the task element Z1 is a dangerous task, the process may proceed to step S06. In this case, if the labor time for the task Z or the task element Z1 is less than the predetermined time and if the task Z or the task element Z1 is not a dangerous task, the process proceeds to step S07.

In another example, if the labor time for the task Z or the task element Z1 is equal to or longer than the predetermined time and if the task Z or the task element Z1 is a dangerous task, the process may proceed to step S06, or, otherwise, the process may proceed to step S07.

In step S07, the processor 26 determines whether the person A3 is in poor physical condition. For example, the processor 26 transmits, to the terminal apparatus 16 of the person A3, a message inquiring whether the person A3 is in poor physical condition. The message is displayed on the terminal apparatus 16. The person A3 operates the terminal apparatus 16 and responds to the inquiry as to whether the person A3 is in poor physical condition. Information indicating the response is transmitted from the terminal apparatus 16 of the person A3 to the control apparatus 10, and the processor 26 determines, based on the response, whether the person A3 is in poor physical condition.

In another example, the processor 26 may observe the person A3 by using the camera or any other sensor to determine whether the person A3 is in poor physical condition. For example, the processor 26 may use a temperature sensor, a sphygmomanometer, or various other sensors to measure the body temperature, blood pressure, pulse, heart rate, blood glucose level, or the like of the person A3, and determine whether the person A3 is in poor physical condition. Alternatively, the processor 26 may cause the camera to capture an image of the person A3 and determine, based on the behavior, attitude, or the like of the person A3, whether the person A3 is in poor physical condition. For example, behaviors, attitudes, or the like of a person who is fatigued may be defined in advance, and the processor 26 may determine whether the person A3 is in poor physical condition in accordance with the definitions.

If it is determined that the person A3 is in poor physical condition (Yes in S07), the processor 26 performs a process for treating the person A3 with poor physical condition. For example, the processor 26 instructs the AI (B3) or the robot C3 to inform the medical institution that the person A3 is in poor physical condition, or the processor 26 contacts the medical institution (S08). In another example, the processor 26 may instruct the AI (B3) or the robot C3 to encourage the person A3 to rest, or transmit a message to the terminal apparatus 16 of the person A3 to encourage the person A3 to rest. The message is displayed on the terminal apparatus 16. In this way, when the person A3 is in poor physical condition, the processor 26 performs processing not based on the determination of the person A3. The processing performed here is processing for treating a person with poor physical condition.

The processing of step S08 is performed, for example, when the task Z or the task element Z1 is a physical task, the labor time is less than the predetermined time, and the person A3 is not performing the task element Z1 and is in poor physical condition. In another example, the processing of step S08 is performed when the task Z or the task element Z1 is a physical task and is not a dangerous task and the person A3 is not performing the task element Z1 and is in poor physical condition. That is, if the person A3 is not performing the task element Z1 although the labor time is not long or the task Z or the task element Z1 is not a dangerous task, it is determined whether the person A3 is in poor physical condition. If the person A3 is in poor physical condition, the processing of step S08 is performed. That is, the processor 26 performs processing not based on the determination of the person A3.

If it is determined that the person A3 is not in poor physical condition (No in S07), the processor 26 prompts the person A3 to perform the task element Z1 (S09). For example, the processor 26 instructs the AI (B3) or the robot C3 to advise the person A3 to perform the task element Z1. Upon receipt of the instruction, for example, the robot C3 may move to the person A3 and generate audio toward the person A3 to perform the task element Z1. The processor 26 may transmit a message prompting the person A3 to perform the task element Z1 to the terminal apparatus 16 of the person A3. The message is displayed on the terminal apparatus 16. In this way, when the person A3 is not in poor physical condition, the processor 26 performs processing not based on the determination of the person A3. The processing performed here is processing for prompting the person A3 to perform the task element Z1. In another example, the processor 26 may cause the AI (B3) or the robot C3 to perform the task element Z1 on behalf of the person A3. That is, the processor 26 may instruct the AI (B3) or the robot C3 to perform the task element Z1.

The processing of step S09 is performed, for example, when the task Z or the task element Z1 is a physical task, the labor time is less than the predetermined time, and the person A3 is not performing the task element Z1 and is not in poor physical condition. In another example, the processing of step S09 is performed when the task Z or the task element Z1 is a physical task and is not a dangerous task and the person A3 is not performing the task element Z1 and is not in poor physical condition. That is, if the person A3 is not performing the task element Z1 although the labor time is not long or the task Z or the task element Z1 is not a dangerous task, it is determined whether the person A3 is in poor physical condition. If the person A3 is not in poor physical condition, the processing of step S09 is performed. That is, the processor 26 performs processing not based on the determination of the person A3.

If the task Z is not a physical task (No in S03), the process proceeds to step S05. The following describes the processing of step S05 and the processing after step S05.

For example, if the task Z, which is an intellectual task, is a task of interacting with any other person, such as a negotiation, a conference, a meeting, the generation of an electronic mail, or posting to an SNS (Yes in S05), the processor 26 causes the person A3 to continue the current behavior (S06). If the task element Z1 assigned to the person A3 in the task Z is a task of interacting with any other person, the processor 26 causes the person A3 to continue the current behavior.

Since the person A3 is not performing the task element Z1 at this stage, the person A3 does not still perform the task element Z1. That is, the processor 26 does not prompt the person A3 to perform the task element Z1. If the task Z is an intellectual task and is a task of interacting with any other person, such as a negotiation, the person A3 may not have performed the task element Z1 for reason such as taking precedence over negotiation or putting pressure on negotiation. Accordingly, the processor 26 causes the person A3 to continue the current behavior with consideration of the intention or behavior of the person A3.

If the task Z, which is an intellectual task, is not a task of interacting with any other person, such as a negotiation, a conference, a meeting, the generation of an electronic mail, or posting to an SNS (No in S05), the process proceeds to step S07. The process may proceed to step S07 when the task element Z1 assigned to the person A3 is not a task of interacting with any other person.

As described above, in step S07, the processor 26 determines whether the person A3 is in poor physical condition.

If it is determined that the person A3 is in poor physical condition (Yes in S07), as described above, the processor 26 performs processing for treating the person A3 with poor physical condition (S08). In this way, if the person A3 is in poor physical condition, the processor 26 performs processing not based on the determination of the person A3.

The processing of step S08 is performed, for example, when the task Z or the task element Z1 is an intellectual task and is not a task of interacting with any other person, such as a negotiation, and the person A3 is not performing the task element Z1 and is in poor physical condition. That is, if the person A3 is not performing the task element Z1 although the task Z or the task element Z1 is not a task of interacting with any other person, such as a negotiation, it is determined whether the person A3 is in poor physical condition. If the person A3 is in poor physical condition, the processing of step S08 is performed. That is, the processor 26 performs processing not based on the determination of the person A3.

If it is determined that the person A3 is not in poor physical condition (No in S07), as described above, the processor 26 prompts the person A3 to perform the task element Z1 (S09).

The processing of step S09 is performed, for example, when the task Z or the task element Z1 is an intellectual task and is not a task of interacting with any other person, such as a negotiation, and the person A3 is not performing the task element Z1 and is not in poor physical condition. That is, the processor 26 performs processing not based on the determination of the person A3.

The process illustrated in the flowchart described above may be repeatedly performed, and the results of the process may be utilized for the assignment of tasks to the person 18, labor management, human resources management, or the like. For example, if the person 18 does not perform the task although the person 18 is prompted to perform the task in the processing of step S09, the result may be reflected in human resources management or the like.

The processor 26 may perform processing based on or not based on the determination of the person 18 in accordance with the attributes of the person 18. The attributes of the person 18 include, for example, the ability of the person 18 for the task, the skill of the person 18, the pay status (such as salary or remuneration) of the person 18, the position of the person 18 in the workplace, and the motivation, emotion, feeling, and the like of the person 18.

Information indicating the attributes of each person 18 may be stored in the control apparatus 10 or any other device. For example, information indicating the skill or ability of each person 18, information indicating the pay status, information indicating the position, and the like are stored in the control apparatus 10 or any other device in advance. Alternatively, the person 18 may operate the terminal apparatus 16 to input information on the current motivation, emotion, feeling, and the like, and the processor 26 may acquire the information from the terminal apparatus 16.

For example, in step S04 described above, the processor 26 may determine, based on the skill or ability of the person A3 and the task element Z1 assigned to the person A3, whether the task element Z1, which is a physical task, is dangerous for the person A3 having the skill or the ability. For example, for each task element, an ability or skill to accomplish the task element without danger is determined, and information indicating the ability or skill is stored in the control apparatus 10, any other device, or the like. The processor 26 refers to the information to determine whether the task element Z1 is dangerous for the person A3. Accordingly, it may be possible to determine, for each person 18, whether a task assigned to the person 18 is a dangerous task.

If it is determined, based on the skill or ability of the person A3, that the task element Z1 is dangerous for the person A3, the processor 26 may cancel the assignment of the task element Z1 to the person A3 and assign the task element Z1 to a person 18 other than the person A3.

In step S05 described above, the processor 26 may determine, based on the skill or ability of the person A3 and the task element Z1 assigned to the person A3, whether the person A3 having the skill or the ability is able to accomplish the task element Z1, which is an intellectual task, without any problem. For example, for each task element, an ability or skill to accomplish the task element without any problem is determined, and information indicating the ability or skill is stored in the control apparatus 10, any other device, or the like. The processor 26 refers to the information to determine whether the task element Z1 can be accomplished by the person A3 without any problem. Accordingly, it may be possible to determine, for each person 18, whether the person 18 is able to accomplish the assigned task without any problem.

In another example, the processor 26 may determine, based on the skill or ability of the person 18 and the task element assigned to the person 18, whether the task element is error prone for the person 18. Here, the task element may be a physical task or an intellectual task. If the assigned task element is error prone for the person 18, the processor 26 may cancel the assignment of the task element to the person 18 and assign the task element to any other person 18. For example, for each task element, a skill or ability to accurately perform the task element is determined, and information indicating the skill or ability is stored in the control apparatus 10 or any other device. The processor 26 refers to the information to determine whether the task element assigned to the person 18 is error prone for the person 18.

If the result of performing the task Z is a predetermined good result, the processor 26 may cause the AI (B3) or the robot C3 to learn the result. For example, the processor 26 transmits information indicating the result to the AI (B3) or the robot C3 and instructs the AI (B3) or the robot C3 to learn the result to reproduce the result. Upon receipt of the instruction, the AI (B3) or the robot C3 learns the result, the task element or other processing performed to obtain the result, and the like.

Whether the result of performing the task Z is good may be determined by the person A3, who is the task operator of the task Z, or by the AI (B3) or the robot C3.

In another example, the processor 26 may manage a history of the results of performing individual tasks and store information indicating the history in the control apparatus 10 or any other device. For example, the information indicating the history includes information indicating whether the result of performing each task is good. The processor 26 may refer to the information indicating the history to determine whether the result of performing the task Z is good.

In one example, when the task Z is a physical task of transporting an object and the object is successfully transported within a task time, it is determined that the result of performing the task Z is good. In another example, when the task Z is an intellectual task, namely, a negotiation, and the negotiation is successful, it is determined that the result of performing the task Z is good. In this case, the processor 26 causes the AI (B3) or the robot C3 to learn the result of the task Z, the task element or other processing performed to obtain the result, and the like.

If the task Z is a predetermined urgent task, the processor 26 may prompt the person A3 to perform the task Z regardless of the evaluation of the person A3. Also when the task element Z1 is a predetermined urgent task, the processor 26 may prompt the person A3 to perform the task element Z1 regardless of the evaluation of the person A3 for the task element Z1. Examples of the urgent task include a potentially lethal task, a task with deadline, and a task with close deadline. Other tasks may be defined as urgent tasks. Urgent tasks are determined in advance, and information indicating the urgent tasks is stored in the control apparatus 10 or any other device. The processor 26 refers to the information to determine whether the task Z or the task element Z1 is an urgent task. For example, even when the person A3 is resting, the processor 26 prompts the person A3 to perform the task element Z1.

If the person A3 is not suitable for performing the task element Z1, which is an urgent task, the processor 26 may cancel the assignment of the task element Z1 to the person A3 and assign the task element Z1 to a person 18 other than the person A3. That is, the processor 26 changes the person 18 to whom the task element Z1 is to be assigned. For example, if the skill or ability of the person A3 is not sufficient to complete the task element Z1 within a predetermined time, the processor 26 may assign the task element Z1 to any other person 18 having skill or ability sufficient to complete the task element Z1 within the predetermined time.

The learning result of an urgent task may be immediately passed to an external device and applied to the external device. For example, in case of environmental threats such as virus or toxic gas invisible to the human eye and harmful to the human body, AI or a robot may immediately instruct a person working in the poor environment to interrupt working, guide the person to a safe location, take over a task that the person would be scheduled to perform, or encourage the person to wear an outfit suitable for the environment before performing the task.

For example, for each task element, information indicating the task element, information indicating the skill or ability, and information indicating the time taken for the person 18 having the skill or the ability to complete the task element are stored in the control apparatus 10 or any other device in association with each other. The processor 26 refers to the information to determine whether the task element Z1 is completed within a predetermined time by using the skill or ability of the person A3.

In the example illustrated in FIG. 4, a task is assigned to the person 18, the AI 12, and the robot 14. Alternatively, a task may be assigned to the person 18 and at least one of the AI 12 and the robot 14.

In the embodiments above, the term “processor” refers to hardware in a broad sense. Examples of the processor include general processors (e.g., CPU: Central Processing Unit) and dedicated processors (e.g., GPU: Graphics Processing Unit, ASIC: Application Specific Integrated Circuit, FPGA: Field Programmable Gate Array, and programmable logic device).

In the embodiments above, the term “processor” is broad enough to encompass one processor or plural processors in collaboration which are located physically apart from each other but may work cooperatively. The order of operations of the processor is not limited to one described in the embodiments above, and may be changed.

The foregoing description of the exemplary embodiments of the present disclosure has been provided for the purposes of illustration and description. It is not intended to be exhaustive or to limit the disclosure to the precise forms disclosed. Obviously, many modifications and variations will be apparent to practitioners skilled in the art. The embodiments were chosen and described in order to best explain the principles of the disclosure and its practical applications, thereby enabling others skilled in the art to understand the disclosure for various embodiments and with the various modifications as are suited to the particular use contemplated. It is intended that the scope of the disclosure be defined by the following claims and their equivalents.

What is claimed is:

1. An information processing apparatus comprising:
a processor configured to
change an index of evaluation of a person for a task assigned to the person, depending on whether the task is a physical task or an intellectual task, and
perform processing based on a determination of the person when the evaluation is evaluation of a non-physical condition of the person or is other than evaluation indicating that the person is not in poor physical condition and is failing to perform the task.

2. The information processing apparatus according to claim 1, wherein
the processor is configured to
perform processing not based on the determination of the person when the evaluation using the index indicates that the person is in poor physical condition or indicates that the person is not in poor physical condition and is failing to perform the task.

3. The information processing apparatus according to claim 1, wherein
the processor is configured to
perform processing based on the determination of the person when the task is a physical task and a labor time for the task is equal to or longer than a predetermined time.

4. The information processing apparatus according to claim 1, wherein
the processor is configured to
prompt the person to perform the task when the task is a physical task, a labor time for the task is less than a predetermined time, and the evaluation does not indicate poor physical condition.

5. The information processing apparatus according to claim 1, wherein
the processor is configured to
perform processing based on the determination of the person when the task is a physical task and is a predetermined dangerous task.

6. The information processing apparatus according to claim 1, wherein
the processor is configured to
prompt the person to perform the task when the task is a physical task and is not a predetermined dangerous task and the evaluation does not indicate poor physical condition.

7. The information processing apparatus according to claim 1, wherein
the processor is configured to
perform processing based on or not based on the determination of the person in accordance with an attribute of the person.

8. The information processing apparatus according to claim 1, wherein
the processor is configured to
perform processing based on the determination of the person when the task is an intellectual task and is a task of interacting with another person other than the person.

9. The information processing apparatus according to claim 1, wherein
the processor is configured to
prompt the person to perform the task when the task is an intellectual task and is not a task of interacting with another person other than the person and the evaluation does not indicate poor physical condition.

10. The information processing apparatus according to claim 1, wherein
the task is further assigned to at least one of an artificial intelligence or a robot as a task to be performed in cooperation with the person, and
the processor is configured to
cause at least one of the artificial intelligence or the robot to learn a result of the task when the result of the task is a predetermined good result.

11. The information processing apparatus according to claim 2, wherein
the task is further assigned to at least one of an artificial intelligence or a robot as a task to be performed in cooperation with the person, and
the processor is configured to
cause at least one of the artificial intelligence or the robot to learn a result of the task when the result of the task is a predetermined good result.

12. The information processing apparatus according to claim 3, wherein
the task is further assigned to at least one of an artificial intelligence or a robot as a task to be performed in cooperation with the person, and
the processor is configured to
cause at least one of the artificial intelligence or the robot to learn a result of the task when the result of the task is a predetermined good result.

13. The information processing apparatus according to claim 4, wherein
the task is further assigned to at least one of an artificial intelligence or a robot as a task to be performed in cooperation with the person, and
the processor is configured to
cause at least one of the artificial intelligence or the robot to learn a result of the task when the result of the task is a predetermined good result.

14. The information processing apparatus according to claim 5, wherein
the task is further assigned to at least one of an artificial intelligence or a robot as a task to be performed in cooperation with the person, and
the processor is configured to
cause at least one of the artificial intelligence or the robot to learn a result of the task when the result of the task is a predetermined good result.

15. The information processing apparatus according to claim 6, wherein
the task is further assigned to at least one of an artificial intelligence or a robot as a task to be performed in cooperation with the person, and
the processor is configured to
cause at least one of the artificial intelligence or the robot to learn a result of the task when the result of the task is a predetermined good result.

16. The information processing apparatus according to claim 7, wherein
the task is further assigned to at least one of an artificial intelligence or a robot as a task to be performed in cooperation with the person, and
the processor is configured to
cause at least one of the artificial intelligence or the robot to learn a result of the task when the result of the task is a predetermined good result.

17. The information processing apparatus according to claim 1, wherein
the processor is configured to
prompt the person to perform the task regardless of the evaluation when the task is a predetermined urgent task.

18. The information processing apparatus according to claim 1, wherein
the processor is configured to
assign the task to another person different from the person when the task is a predetermined urgent task and the person is not suitable for the task.

19. The information processing apparatus according to claim 1, wherein
the evaluation is obtained in response to the person failing to perform the task.

20. A non-transitory computer readable medium storing a program causing a computer to execute a process for information processing, the process comprising:
changing an index of evaluation of a person for a task assigned to the person, depending on whether the task is a physical task or an intellectual task; and
performing processing based on a determination of the person when the evaluation is evaluation of a non-physical condition of the person or is other than evaluation indicating that the person is not in poor physical condition and is failing to perform the task.
